(19) United States
(12) Patent Application Publication
(10) Pub. No.: US 2026/0027363 A1
(43) Pub. Date: Jan. 29, 2026

(54) VESTIBULAR ELECTRICAL STIMULATION APPARATUS, CONTROL SYSTEM, AND CONTROL METHOD

(71) Applicant: Murata Manufacturing Co., Ltd., Nagaokakyo-shi (JP)

(72) Inventors: Kazuki MATSUI, Nagaokakyo-shi (JP); Jian LUAN, Nagaokakyo-shi (JP); Kazuma AOYAMA, Maebashi-shi (JP)

(73) Assignee: Murata Manufacturing Co., Ltd., Nagaokakyo-shi (JP)

(21) Appl. No.: 19/346,630

(22) Filed: Oct. 1, 2025

Related U.S. Application Data
(63) Continuation of application No. PCT/JP2024/013210, filed on Mar. 29, 2024.

(30) Foreign Application Priority Data
Apr. 27, 2023 (JP) 2023-072929

(51) Int. Cl. A61N 1/36; A63F 13/25

(52) U.S. Cl. CPC A61N 1/36025; A61N 1/36034; A63F 13/25; A61N 1/36036

(57) ABSTRACT

A vestibular electrical stimulation apparatus includes an electrode configured to provide electrical stimulation to a vestibular organ of a user, an output circuit configured to apply a current to the electrode, a control circuit configured to control the current to be applied by the output circuit, and an input circuit configured to receive a control signal from an external apparatus. The control circuit is configured to apply a first current, which is a noise current, based on the control signal received by the input circuit.

### CROSS-REFERENCE TO RELATED APPLICATIONS

The present application bypass continuation of International Application PCT/JP2024/013210, filed Mar. 29, 2024, and 2023-072929, filed Apr. 27, 2023, the entire contents of each of which being incorporated herein by reference.

### TECHNICAL FIELD

The present disclosure relates to a vestibular electrical stimulation apparatus, a control system, and a control method.

### BACKGROUND ART

“Motion sickness” of which symptoms such as nausea, vomiting, facial pallor, and cold sweat are caused by riding in conveyances such as a vehicle, a train, a ship, and an airplane is widely known. Motion sickness may develop not only during rides in conveyances but also during a play of VR (Virtual Reality) games with VR goggles on. Methods for recovering from motion sickness include resting in a motionless state, which is a common recovery method. Oral medications for suppressing the symptoms of motion sickness are also widely available on the market.

The cause of motion sickness is considered to be related to vision, a vestibular sensation produced by the vestibular organ, and a somatic sensation. Japanese Unexamined Patent Application Publication No. 2006-288665 (Patent Document 1) describes a method that uses vestibular electrical stimulation (GVS: Galvanic Vestibular Stimulation) to suppress the development of motion sickness in a user riding in a ship. The vestibular electrical stimulation refers to a method of applying electrical stimulation to the vestibular organ of a user to make the user experience pseudo-accelerations. Patent Document 1 describes applying a current between a pair of electrodes attached to the head of a user to provide the user with an acceleration sensation in a direction for cancelling out a ship motion.

### CITATION LIST

### Patent Document

Patent Document 1: Japanese Unexamined Patent Application Publication No. 2006-288665

### SUMMARY

### Technical Problems

However, in the method that uses vestibular electrical stimulation to suppress the symptoms of motion sickness as described in Patent Document 1, a pseudo-vestibular sensation (acceleration sensation) needs to be provided to the user at an appropriate timing and an appropriate magnitude to cancel out the ship motion. If the user is provided with a pseudo-vestibular sensation at a timing and a magnitude that are different from the timing and magnitude that cancel out the ship motion, the symptoms of motion sickness may worsen. Therefore, an electrical stimulation apparatus that can alleviate the symptoms of motion sickness without worsening the symptoms of motion sickness is desired.

The present disclosure has been made to solve such an issue, and is directed to providing a vestibular electrical stimulation apparatus that provides electrical stimulation to the vestibular organ of a user and can alleviate the symptoms of motion sickness.

### Solutions to Problems

A vestibular electrical stimulation apparatus according to the present disclosure includes an electrode, an output circuit, a control circuit, and an input circuit. The electrode provides electrical stimulation to a vestibular organ of a user. The output circuit applies a current to the electrode. The control circuit controls the current to be applied by the output circuit. The input circuit receives a control signal from an external apparatus, e.g., a game console, a computing device, or a vehicle control system. The control circuit applies a first current, based on the control signal received by the input circuit. The first current is a noise current.

### Advantageous Effects

According to the present disclosure, the vestibular electrical stimulation apparatus that provides electrical stimulation to the vestibular organ of the user applies the first current, which is the noise current, to the user to successfully alleviate the symptoms of motion sickness.

### BRIEF DESCRIPTION OF DRAWINGS

FIG. 1 is a schematic diagram of a control system according to a first embodiment.

FIG. 2 is an external view of goggles including electrodes and a display device.

FIG. 3 is a diagram for describing an operation of the control system according to the first embodiment.

FIG. 4 is a diagram for describing a prediction of the brain and sensory inputs that occur during a play of a VR game.

FIG. 5 is a diagram for describing a prediction of the brain and sensory inputs that occur in the case where a noise current is applied.

FIG. 6 is a diagram for describing the noise current.

FIG. 7 is a diagram illustrating a current pattern to be applied to electrodes by a vestibular electrical stimulation apparatus according to the first embodiment.

FIG. 8 is a flowchart for describing an operation of the vestibular electrical stimulation apparatus according to the embodiment.

FIG. 9 is a schematic diagram of a vestibular electrical stimulation apparatus in a second embodiment.

FIG. 10 is a diagram for describing a current less than a perception threshold and a current greater than or equal to the perception threshold in the second embodiment.

FIG. 11 is a schematic diagram of a control system in a third embodiment.

### DESCRIPTION OF EMBODIMENTS

Embodiments will be described in detail below with reference to the drawings. In the drawings, the same or corresponding parts are denoted by the same reference signs, and the description thereof will not be repeated.

### FIRST EMBODIMENT

### Overview of Control System

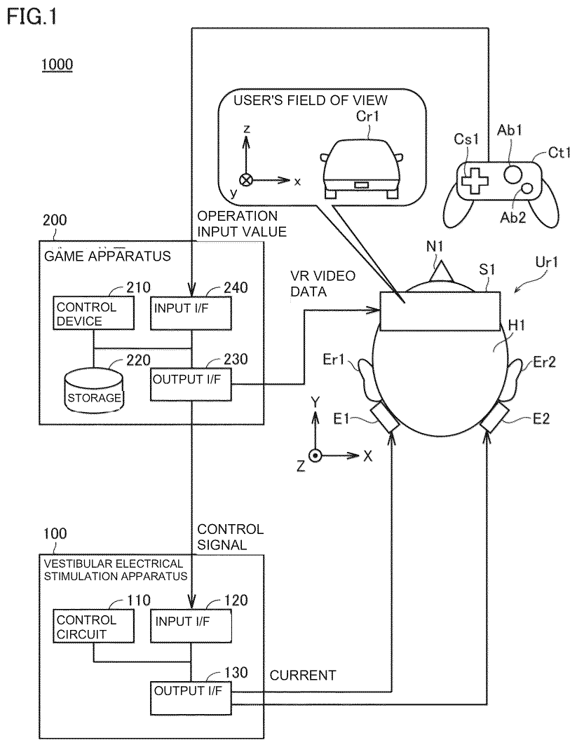

FIG. 1 is a schematic diagram of a control system 1000 according to a first embodiment. The control system 1000 is a game system that uses a vestibular electrical stimulation apparatus 100 and a game apparatus 200 (external apparatus) in combination to make a user Ur1 who plays a game with the game apparatus 200 feel a vestibular sensation in a pseudo manner with the vestibular electrical stimulation apparatus 100. The game apparatus 200 described below is a VR game apparatus that shows a virtual space to the user Ur1 by using a head-mounted display (HMD) and allows the user Ur1 to operate an object (e.g., Cr1) in the virtual space.

By outputting a control signal to the vestibular electrical stimulation apparatus 100, the game apparatus 200 can make, using the vestibular electrical stimulation apparatus 100, the user Ur1 feel pseudo-accelerations produced on an object in accordance with the object that moves in the virtual space. As a result, compared with a simple VR game apparatus, the game apparatus 200 can further increase the user Ur1's sense of immersion into the game by making the user Ur1 feel the pseudo-accelerations produced on the object. Note that the game apparatus 200 is not limited to a VR game apparatus that uses a head-mounted display, and may be a game apparatus that uses a two-dimensional display such as a liquid crystal display or an organic EL display.

The vestibular electrical stimulation apparatus 100 includes electrodes E1 and E2, a control circuit 110, an input interface 120, and an output interface 130. In the vestibular electrical stimulation apparatus 100, the electrodes E1 and E2 are attached to the user Ur1 as illustrated in FIG. 1. Note that FIG. 1 illustrates the user Ur1 viewed from above, and illustrates a head H1, a left ear Er1, a right ear Er2, and a nose N1 of the user Ur1. In the first embodiment, an example of the vestibular electrical stimulation apparatus 100 including two electrodes, i.e., the electrodes E1 and E2, will be described. However, in a certain aspect, the number of electrodes included in the vestibular electrical stimulation apparatus 100 may be one, or three or more.

FIG. 1 illustrates X, Y, and Z axes representing three-axis directions of a real space where the user Ur1 is present. Among the X, Y, and Z axes, a vertical direction with respect to a floor surface on which the user Ur1 is standing is defined as a “Z-axis direction”, a direction that is perpendicular to the Z-axis direction and to which the user Ur1 faces is defined as a “Y-axis direction”, and a direction that is perpendicular to both the Y-axis direction and the Z-axis direction is defined as an “X-axis direction”. A positive Z-axis direction may be referred to as an upper side. A negative Z-axis direction may be referred to as a lower side. A positive Y-axis direction may be referred to as a front side. A negative Y-axis direction may be referred to as a rear side. A positive X-axis direction may be referred to as a right side. A negative X-axis direction may be referred to as a left side.

The vestibular electrical stimulation apparatus 100 is an apparatus that provides electrical stimulation to the vestibular organ of the user Ur1 to produce a pseudo-vestibular sensation in the user Ur1. The vestibular sensation is a sensation that the user Ur1 can sense with the vestibular organ. The vestibular organ includes hair cells that are in the semicircular canals and in the utricle and saccule of the otolith organ. The semicircular canals receive rotational angular accelerations with respect to the three axes, while the utricle and saccule receive linear accelerations. The vestibular electrical stimulation apparatus 100 applies a current from the output interface 130 to the electrodes E1 and E2 attached to the user Ur1, and thus can provide the user Ur1 with a vestibular sensation corresponding to the current value.

The control circuit 110 is, for example, a hard-wired circuit such as an ASIC (Application Specific Integrated Circuit) or an FPGA (Field-Programmable Gate Array). The control circuit 110 may include a memory such as a RAM (Random Access Memory) and a CPU (Central Processing Unit).

The input interface 120 is connected to the game apparatus 200. The vestibular electrical stimulation apparatus 100 receives a control signal from the game apparatus 200 via the input interface 120. The control signal includes electrical stimulation data or the like. The electrical stimulation data is data including information such as a target value, a polarity, and an application period of the current. Based on the input control signal, the control circuit 110 calculates a value of the current to be applied to the electrodes E1 and E2. The output interface 130 is connected to the electrodes E1 and E2 by wires, and applies the current having the value calculated by the control circuit 110 to the electrodes E1 and E2.

The game apparatus 200 includes a display device S1, a controller Ct1 for games, a control device 210, a storage 220, an output interface 230, and an input interface 240. In the first embodiment, the description will be given below on the assumption that a racing game for driving a car is executed on the game apparatus 200.

The display device S1, which is a head-mounted display, displays VR video data such that the rear portion of a car Cr1 is present at the center of the field of view of the user Ur1. Obviously, the display device S1 may display VR video data from the first-person perspective of the user Ur1 who is riding in the car Cr1. As illustrated in FIG. 1, the display device S1 also displays x, y, and z axes. The x, y, and z axes displayed on the display device S1 represent the three-axis directions in the virtual space. The three-axis directions in the virtual space correspond to the three-axis directions in the real space. A positive z-axis direction is referred to as an upper side. A negative z-axis direction is referred to as a lower side. A positive y-axis direction is referred to as a front side. A negative y-axis direction is referred to as a rear side. A positive x-axis direction is referred to as a right side. A negative x-axis direction is referred to as a left side. The car Cr1 is traveling straight in the positive y-axis direction.

The controller Ct1 at least includes a cross control Cs1 and buttons Ab1 and Ab2. The cross control Cs1 is a control for operating the position of the car Cr1 (object) to be operated by the user Ur1, and may be a key or button of another form such as a stick-shaped input device. The cross control Cs1 may be a gesture input device, an input device having the shape of a steering wheel, or the like. The user Ur1 operates the controller Ct1, and thus can move the car Cr1 displayed in front of them forward or change the traveling direction of the car Cr1.

More specifically, the user Ur1 presses the button Ab1 to move the car Cr1 forward. The user Ur1 presses a right button of the cross control Cs1 to turn the car Cr1 to the right, and presses a left button of the cross control Cs1 to turn the car Cr1 to the left. The left and right buttons of the cross control Cs1 correspond to the steering wheel of a car in the real space. The button Ab1 corresponds to the accelerator of the car in the real space. The button Ab2 corresponds to the brake of the car in the real space. An operation performed by the user Ur1 on the controller Ct1 is input as an operation input value to the control device 210 via the input interface 240. The input interface 240 corresponds to an “operation circuit” of the game apparatus 200.

The control device 210 includes, for example, a processor such as a CPU or an MPU that executes a game program, and a memory such as a RAM. The control device 210 may be a hard-wired circuit such as an ASIC or an FPGA.

The storage 220 is implemented by a rewritable nonvolatile memory or magnetic disk such as an HDD (Hard Disk Drive), an SSD (Solid State Drive), or a flash memory. The storage 220 stores a program of the racing game of the car Cr1 illustrated in FIG. 1. Based on the program, the control device 210 causes the display device S1 to display the car Cr1, which is an object. Specifically, the control device 210 sends VR video data to the display device S1 via the output interface 230.

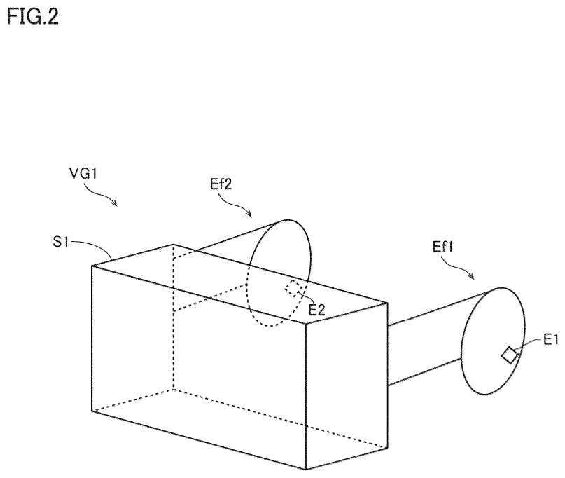

FIG. 2 is an external view of goggles VG1 including the electrodes E1 and E2 and the display device S1. FIG. 2 illustrates the goggles VG1 for VR. The goggles VG1 are a so-called head-mounted display and includes supports Ef1 and Ef2 and the display device S1. The support Ef1 is fixed to the left ear Er1. The support Ef2 is fixed to the right ear Er2. Thus, the goggles VG1 are fixed to the head H1 of the user Ur1 such that the display device S1 covers both eyes of the user Ur1.

The electrodes E1 and E2 are respectively installed on the supports Ef1 and Ef2 to be in contact with the respective mastoid processes of the user Ur1. The electrodes E1 and E2 and the display device S1 may be installed on different fixtures. For example, the electrodes E1 and E2 may be installed on respective earmuffs, and the display device S1 may be smart glasses or the like. The electrodes E1 and E2 need not be installed on fixtures. For example, the electrodes E1 and E2 may have an adhesive force from a hydrogel or the like on the surfaces thereof, and may be directly adhered to the respective mastoid processes.

The positions to which the electrodes E1 and E2 are adhered are not limited to the mastoid processes, and may be positions that are non-hairy areas free from hair, beard, or the like. As illustrated in FIG. 2, in the vestibular electrical stimulation apparatus 100 according to the first embodiment, the electrodes E1 and E2 and the display device S1 are integrally formed in the goggles VG1. This can simplify the work of attaching the electrodes E1 and E2 to the user Ur1.

The goggles VG1 illustrated in FIG. 2 may include the components included in the vestibular electrical stimulation apparatus 100 and the game apparatus 200 illustrated in FIG. 1. Specifically, the goggles VG1 may be configured as a head-mounted display into which the game apparatus 200 and the vestibular electrical stimulation apparatus 100 are integrated. In this case, the goggles VG1 include a single control device. The control device has the functions of both the control device 210 of the game apparatus 200 and the control circuit 110 of the vestibular electrical stimulation apparatus 100.

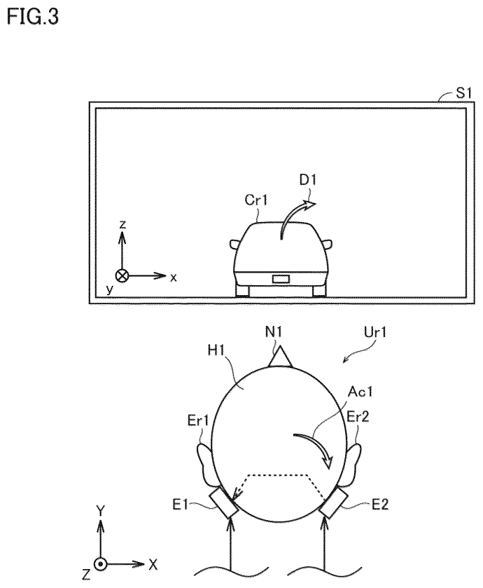

An operation of the control system 1000 will be described next. FIG. 3 is a diagram for describing the operation of the control system 1000 according to the first embodiment. Upon receiving an operation of the user Ur1 for pressing the right button of the cross control Cs1, the game apparatus 200 turns the car Cr1 displayed on the display device S1 in a right direction D1 (the positive x-axis direction). The vestibular electrical stimulation apparatus 100 applies a current to the electrodes E1 and E2 to make the user Ur1 feel an acceleration in the right direction in accordance with the display of the car Cr1 turning in the right direction D1.

Specifically, to evoke a pseudo-vestibular sensation in the user Ur1 so that the user Ur1 can feel turning in the right direction, the vestibular electrical stimulation apparatus 100 applies a current such that the electrode E2 serves as a positive terminal and the electrode E1 serves as a negative terminal. When the current flows from the electrode E1 to the electrode E2, the current flows through the ear canal of the user Ur1 and provides electrical stimulation to the vestibular organ. This can cause a sensation of an acceleration Ac1 in the roll direction in the user Ur1. From the viewpoint of safety, the current that flows between the electrode E1 and the electrode E2 may have a value of 4.0 mA or less.

As described above, the electrodes E1 and E2 are respectively attached to the left and right mastoid processes, so that the vestibular electrical stimulation apparatus 100 according to the first embodiment can provide the user Ur1 with a pseudo-vestibular sensation in the X- axis direction. The vestibular electrical stimulation apparatus 100 may provide the user Ur1 with pseudo-vestibular sensations in the roll, pitch, and yaw directions or in the X-axis, Y-axis, and Z-axis directions, by changing the arrangement of the electrodes E1 and E2 and the number of electrodes.

Thus, with the vestibular electrical stimulation apparatus 100, the control system 1000 can make the user Ur1 feel a pseudo-vestibular sensation of turning in the right direction in accordance with the image of the car Cr1, displayed in the game on the game apparatus 200, turning in the right direction. Therefore, compared with simply showing the user Ur1 an image of a car turning in the right direction in a game, showing the user Ur1 the image of the car turning in the right direction with the vestibular electrical stimulation apparatus 100 providing the user Ur1 with a pseudo-vestibular sensation can increase the user Ur1's sense of immersion into the game. The current perceivable by the user as the pseudo-acceleration as illustrated in FIG. 3 corresponds to a “third current” in the present disclosure.

The user Ur1 who plays games on the game apparatus 200 illustrated in FIGS. 1 to 3 may develop motion sickness.

The cause of motion sickness is considered to be related to the vision, the vestibular sensation, and the somatic sensation of the user Ur1. The somatic sensation is a general term for skin sensations such as senses of touch, pressure, warmth, cold, and pain, and deep sensations that convey the motions or positions of the limbs.

The relationship between the brain and the vision, vestibular sensation, and somatic sensation will be described using the free energy principle, which is an information theory of the brain. The brain functions to predict, based on inputs (vision, vestibular sensation, somatic sensation) from sensory organs, future inputs from the sensory organs and minimize a future prediction error. For example, a user has learned, from their experiences such as actually driving a vehicle, that when the vehicle makes a sharp turn, a vestibular sensation occurs due to the centrifugal force. When a user is actually riding in a vehicle that is making a sharp turn, the user's brain predicts that the vestibular sensation and the somatic sensation will occur, based on vision information of the vehicle making a sharp turn. When the sensory inputs due to the centrifugal force are actually provided to the user as the vestibular sensation and the somatic sensation thereafter, a sensation matching state (with a small prediction error) can be observed.

However, when the user plays VR games, the error between the prediction of the brain and the inputs (vision, vestibular sensation, and somatic sensation) from the sensory organs may become large. For example, the acceleration Ac1 described in FIG. 3 may differ from the vestibular sensation predicted by the brain of the user Ur1.

When a user undergoes such a VR experience of riding in a car, upon receiving vision information of the car making a sharp turn in the VR space, the brain expects (predicts) similar inputs indicating the sharp turn also to the vestibular sensation and the somatic sensation. However, in reality, inputs that would occur in response to a sharp turn (such as a rotation input to the semicircular canals and an acceleration (G) to the somatic sensation) do not occur in the vestibular sensation and the somatic sensation. In general VR games in which no vestibular electrical stimulation is performed, no pseudo-vestibular sensation is provided.

Therefore, an error occurs between the prediction of the brain and the inputs to the sensory organs. The present embodiment assumes that a person having an experience of driving or being aboard a car has learned the sensory inputs that have occurred during the ride. However, the match between the vision and the vestibular sensation and somatic sensation (the scenery which the user sees moves as the user moves) similarly occurs during movement such as walking. It is expected that even a person not having an experience of driving or being aboard a car but has an experience of “moving” may develop the similar symptoms of motion sickness.

When the error caused between the prediction of the brain and the inputs to the sensory organs cannot be solved, the unsolved error is considered to lead to the symptoms of motion sickness. It is considered that the symptoms of motion sickness can be alleviated by preventing the brain of the user Ur1 from recognizing that the error is caused between the prediction and the inputs to the sensory organs.

According to the free energy principle, when there is an error between the prediction and the inputs to the sensory organs, the brain performs feedback processing to minimize the prediction error. For example, even when images of a turning car are acquired as the vision information but no accelerations are provided as the vestibular sensation, the user Ur1 sometimes does not develop motion sickness. This is because, even when there is an error between the prediction of the brain of the user Ur1 and the inputs to the sensory organs, the brain adapts to the error occurred situation by performing the feedback processing, and thus can solve the error caused between the prediction of the brain and the inputs to the sensory organs. In this case, the brain of the user Ur1 can suppress the development of motion sickness by itself. However, the effectiveness of the feedback processing for minimizing the prediction error varies among individuals. That is, even in the same environment, whether the symptoms of motion sickness occur varies depending on individuals.

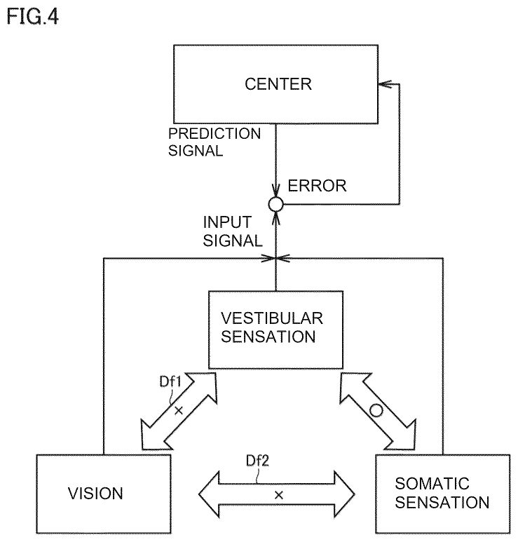

FIG. 4 is a diagram for describing the prediction of the brain and sensory inputs that occur during a play of a VR game. To simplify the description, FIG. 4 illustrates the relationship between the prediction of the brain and sensory inputs that occur when the user Ur1 plays a VR game using only the display device S1 without the application of the current to the electrodes E1 and E2. That is, in FIG. 4, the description will be given on the assumption that the user is playing a VR game with the control system 1000 illustrated in FIGS. 1 to 3 from which the vestibular electrical stimulation apparatus 100 is removed.

“CENTER” illustrated in FIG. 4 means the brain of the user Ur1. As illustrated in FIG. 4, a prediction signal is output from the center. A difference between the prediction signal and an input signal, which is a stimulus provided from each sensory organ, is re-input to the center. The center receives the VR video data as the vision. The center receives, as the vestibular sensation, the state where no vestibular electrical stimulation is performed. The center receives, as the somatic sensation, the motions or positions of the limbs of the user Ur1 during the play of the VR game.

The vision based on the video data displayed by display device S1 deviates from the vestibular sensation and the somatic sensation of the user Ur1 that occur during the play of the VR game. That is, as illustrated in FIG. 4, an error Df1 is caused between a predicted vestibular sensation based on the vision and a predicted actual vestibular sensation. An error Df2 is caused between a predicted somatic sensation based on the vision and an actual somatic sensation. On the other hand, since the vestibular sensation matches the somatic sensation, no error is caused.

In the state illustrated in FIG. 4, the center recognizes two errors illustrated as the errors Df1 and Df2, and attempts to solve these errors Df1 and Df2 by the feedback processing. When the feedback processing does not work effectively and the errors Df1 and Df2 continue to exist, the user Ur1 may develop motion sickness.

If the center gives the vestibular sensation that solves the predicted error Df1 to the user Ur1, the error Df1 is no longer caused between the vestibular sensation and the vision. This leads to the solution to motion sickness. However, the timing and magnitude of the vestibular sensation predicted based on the vision information vary among individuals. This makes it difficult to provide a pseudo-vestibular sensation that completely matches the prediction of the brain of each user from the electrodes E1 and E2. Accordingly, the control system 1000 according to the first embodiment provides a noise current (nGVS: noisy Galvanic Vestibular Stimulation) between the electrode E1 and the electrode E2 to reduce the brain's attention to the vestibular sensation. As used herein, “noise current” refers to an electrical signal with a random or pseudo-random waveform, such as white noise, that does not encode a specific, perceivable sensation of acceleration but rather introduces a baseline level to the user. The noise current may correspond to a “first current” in the present disclosure.

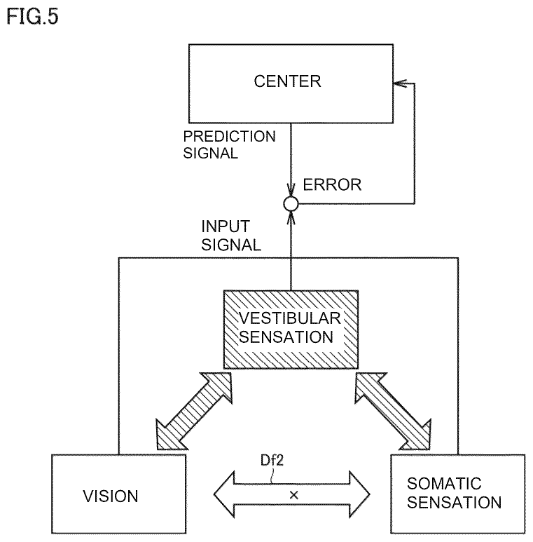

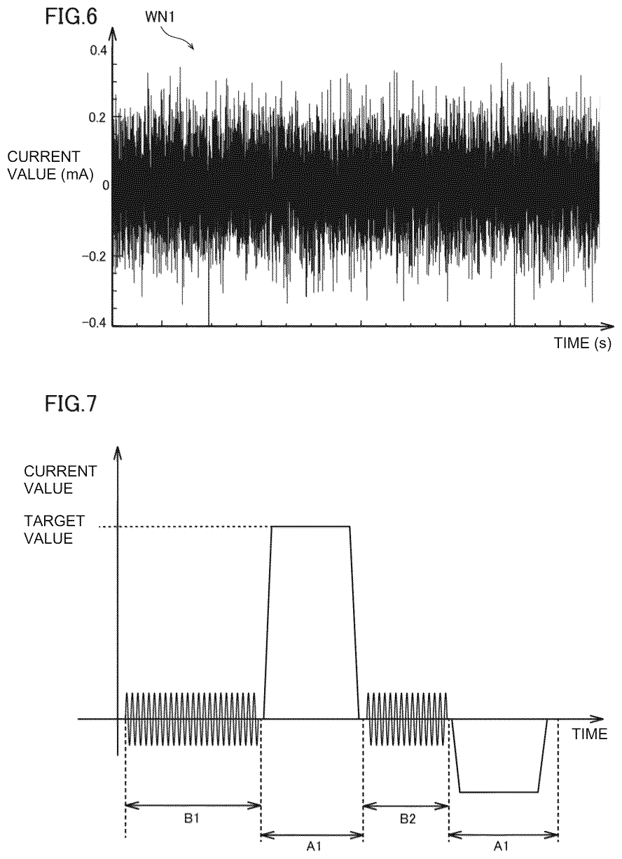

FIG. 5 is a diagram for describing the prediction of the brain and sensory inputs that occur in the case where a noise current is applied. FIG. 6 is a diagram for describing a current WN1, which is the noise current. In the first embodiment, the current WN1, which is a noise current stimulus of a magnitude that is not perceivable by the user Ur1, is applied to the vestibular organ of the user Ur1. When the current WN1 is applied, a noise-like input is applied to the vestibular sensation. This makes the sensation vague, and the input prediction accuracy decreases (the reliability of the vestibular sensation decreases). As a result, the attention of the user Ur1's brain to the vestibular sensation decreases. In other words, due to the application of the current WN1, the function of the user Ur1's brain to perceive the vestibular sensation declines. In the first embodiment, the current WN1, which is the noise current, is a current that is less than a perception threshold of the user Ur1. In another aspect, the current WN1 may be a current that is greater than or equal to the perception threshold of the user Ur1.

Consequently, the function of the user Ur1's brain to sense the error Df1 between the predicted vestibular sensation based on the vision and the actual vestibular sensation declines. Since the ability of the user Ur1's brain to sense the error Df1 declines, the user Ur1's brain senses only the error Df2 as the error between the prediction of the brain and the actual sensory input as illustrated in FIG. 5. Thus, the user Ur1's brain recognizes only the error Df2, and attempts to solve the error Df2 by the feedback processing that is provided as a function of the brain. As compared with FIG. 4, the number of errors between the prediction of the brain and the inputs to the sensory organs reduces from two to one in FIG. 5. That is, in FIG. 5, the number of errors to be solved using the feedback processing is less than that in FIG. 4.

Thus, in the first embodiment, since the number of errors between the prediction of the brain and the inputs to the sensory organs can be reduced, the symptoms of motion sickness can be alleviated. In the example in FIG. 5, the brain just performs the feedback processing for minimizing the error Df2. If the error Df2 can be minimized, motion sickness can be improved by the feedback processing.

The control circuit 110 applies the current WN1 that is less than the perception threshold and illustrated in FIG. 6 to the electrodes E1 and E2. As described above, in the first embodiment, the current WN1 that is less than the perception threshold is the noise current. The current WN1 illustrated in FIG. 6 is white noise with a current density value that is less than 0.5 mA/m².

Since the perception threshold varies depending on the user Ur1, the current density value of the current WN1 may be, for example, 0.4 mA/m². The current WN1 is not limited to a noise current whose center current value is 0 mA, and may be a noise current whose center current value has a positive or negative polarity. For example, when electrical stimulation is provided to the vestibular organ as a current of +0.3 mA, the current WN1 may be a noise current (−0.5 mA to +0.1 mA) whose center current value is −0.2 mA and has a negative polarity.

The current WN1 provided to the user Ur1 may be an alternating current having a predetermined frequency instead of white noise. In this case, for example, the current WN1 has a frequency of 0.1 kHz to 1.0 kHz.

Next, a period for which the vestibular electrical stimulation apparatus 100 applies the current WN1 to the electrodes E1 and E2 will be described. To avoid an influence on electrical stimulation provided to the vestibular organ by GVS, the period in which the vestibular electrical stimulation apparatus 100 applies the current WN1 to the electrodes E1 and E2 may be an operating period of the vestibular electrical stimulation apparatus 100 (e.g., a period from when the game apparatus 200 is turned on to when the game apparatus 200 is turned off) other than a period for which a current for providing electrical stimulation to the vestibular organ is applied to the electrodes E1 and E2. Thus, the current WN1 and the current for providing perceivable electrical stimulation to the vestibular organ are provided in different, non-overlapping periods. FIG. 7 is a diagram illustrating a current pattern to be applied to the electrodes E1 and E2 by the vestibular electrical stimulation apparatus 100 according to the first embodiment. In FIG. 7, the horizontal axis represents the time and the vertical axis represents the current value.

As illustrated in FIG. 7, a period for which the game apparatus 200 is loading the game program (a non-operation period B1 for which the user Ur1 cannot operate the car Cr1) is a period for which the vestibular electrical stimulation apparatus 100 need not provide electrical stimulation to the vestibular organ. Therefore, the vestibular electrical stimulation apparatus 100 applies the current WN1 to the electrodes E1 and E2. In an operation period A1 in which the game is started in the game apparatus 200 and the user Ur1 can operate the car Cr1, the vestibular electrical stimulation apparatus 100 needs to provide electrical stimulation to the vestibular organ in accordance with the operation of the car Cr1. Therefore, the vestibular electrical stimulation apparatus 100 does not apply the current WN1 to the electrodes E1 and E2 to avoid an influence on the electrical stimulation provided to the vestibular organ. While described herein in the context of a video game, the “non-operation period” may be more generally defined as any period in which a user is not intended to perceive a specific, induced vestibular sensation corresponding to an external stimulus. Conversely, an “operation period” is a period where such a specific vestibular sensation is intentionally induced.

A non-operation period B2 for which the user Ur1 cannot operate the car Cr1 because the game apparatus 200 shows game events or displays a menu is a period for which the vestibular electrical stimulation apparatus 100 need not provide electrical stimulation to the vestibular organ. Therefore, the vestibular electrical stimulation apparatus 100 applies the current WN1 to the electrodes E1 and E2. In an operation period A2 in which the game is resumed in the game apparatus 200 and the user Ur1 can operate the car Cr1, the vestibular electrical stimulation apparatus 100 needs to provide electrical stimulation to the vestibular organ in accordance with the operation of the car Cr1. Therefore, the vestibular electrical stimulation apparatus 100 does not apply the current WN1 to the electrodes E1 and E2 to avoid an influence on the electrical stimulation provided to the vestibular organ.

Note that the vestibular electrical stimulation apparatus 100 may apply the current WN1 to the electrodes E1 and E2 even in the operation period in which the game is executed on the game apparatus 200 and the user Ur1 can operate the car Cr1 but the user Ur1 is not operating the car Cr1. More specifically, the setting of the period for which the vestibular electrical stimulation apparatus 100 applies the current WN1 to the electrodes E1 and E2 will be described using a flowchart.

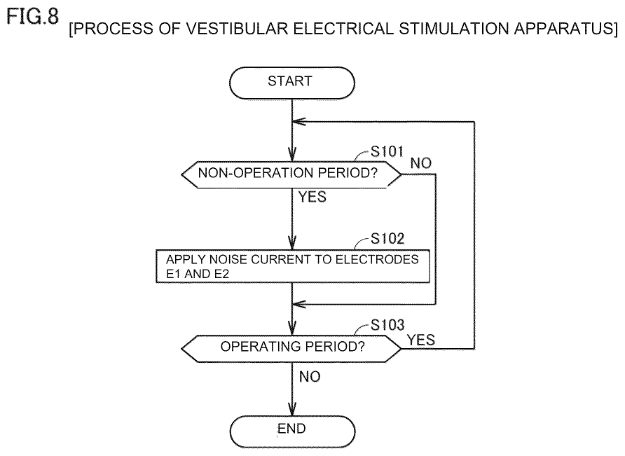

FIG. 8 is a flowchart for describing an operation of the vestibular electrical stimulation apparatus 100 according to the embodiment. First, based on a control signal received from the game apparatus 200 after the game apparatus 200 is turned on, the vestibular electrical stimulation apparatus 100 determines whether a current timing is in the non-operation period in which the user Ur1 cannot operate the car Cr1 (step S101).

The control signal includes information that enables the operation period in which the user Ur1 can operate the car Cr1 and the non-operation period in which the user Ur1 cannot operate the car Cr1 to be distinguished from each other. When the control signal includes information indicating the operation period, the control signal further includes electrical stimulation data that defines the characteristics (such as a target value, a polarity, and an application period) of the current to be applied to the electrodes E1 and E2 in the operation period. The vestibular electrical stimulation apparatus 100 may distinguish between the operation period and the non-operation period, based on whether the control signal includes the electrical stimulation data.

If the current timing is in the non-operation period in which the user Ur1 cannot operate the car Cr1 (YES in step S101), the vestibular electrical stimulation apparatus 100 sets the identified non-operation period as the application period of the current WN1, and applies the current WN1 to the electrodes E1 and E2 (step S102). The non-operation period in the first embodiment may correspond to a “first period” in the present disclosure. The operation period in the first embodiment may correspond to a “second period” in the present disclosure.

The vestibular electrical stimulation apparatus 100 may continuously apply the current WN1 to the electrodes E1 and E2 for the non-operation period. Alternatively, the vestibular electrical stimulation apparatus 100 may stop applying the current WN1 upon an elapse of a certain time (e.g., 60 seconds) even in the non-operation period. The vestibular electrical stimulation apparatus 100 may intermittently apply the current WN1 to the electrodes E1 and E2 in the non-operation period.

If the current timing is in the operation period in which the user Ur1 can operate the car Cr1 (NO in step S101), the vestibular electrical stimulation apparatus 100 skips the processing of step S102. The vestibular electrical stimulation apparatus 100 then determines whether the current timing is in the operating period (step S103). The operating period of the vestibular electrical stimulation apparatus 100 is related to the operating period of the game apparatus 200, and is, for example, a period from when the game apparatus 200 is turned on to when the game apparatus 200 is turned off. Obviously, the operating period of the vestibular electrical stimulation apparatus 100 is not necessarily related to the operating period of the game apparatus 200, and may be, for example, a period from when the vestibular electrical stimulation apparatus 100 is turned on to when the vestibular electrical stimulation apparatus 100 is turned off.

If the current timing is in the operating period (YES in step S103), the vestibular electrical stimulation apparatus 100 returns the process to step S101. On the other hand, if the current timing is not in the operating period (NO in step S103), the vestibular electrical stimulation apparatus 100 ends the process of applying the noise current to the electrodes E1 and E2. Thus, in the first embodiment, by the application of the current WN1 that is less than the perception threshold of the user Ur1 to electrodes E1 and E2 in the non-operation period, the number of errors between the prediction of the brain and the inputs to the sensory organs can be reduced, and the symptoms of motion sickness can be alleviated.

In the first embodiment, the control system 1000 has been described as the game system that uses the vestibular electrical stimulation apparatus 100 and the game apparatus 200 (external apparatus) in combination to make the user Ur1 who plays a game with the game apparatus 200 feel a vestibular sensation in a pseudo manner with the vestibular electrical stimulation apparatus 100. However, the control system 1000 is not limited to the game system, and may be an amusement system that uses the vestibular electrical stimulation apparatus 100 and a video apparatus (external apparatus) in combination to make the user Ur1 feel a vestibular sensation in a pseudo manner with the vestibular electrical stimulation apparatus 100 in accordance with the movement of an object displayed by the video apparatus.

The control system 1000 according to the first embodiment may alleviate the symptoms of motion sickness by providing the current WN1 that is less than the perception threshold before the user Ur1 develops motion sickness. In other words, alleviating the symptoms of motion sickness in this embodiment encompasses both preventing motion sickness and early recovery from motion sickness.

### SECOND EMBODIMENT

In the first embodiment, the configuration of implementing the VR game by the combination of the vestibular electrical stimulation apparatus 100 and the game apparatus 200 has been described. In a second embodiment, a configuration in which the vestibular electrical stimulation apparatus 100 is applied to the user Ur1 riding in a ship will be described.

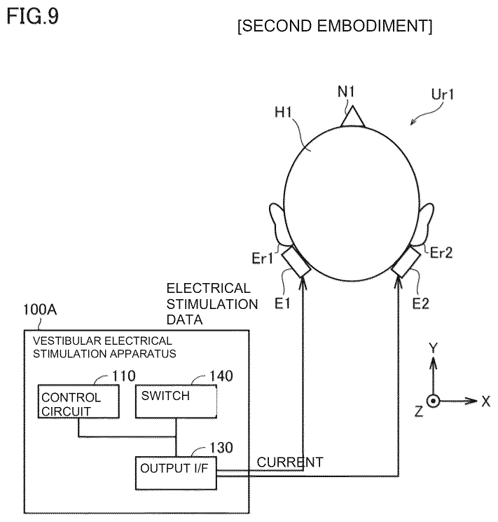

FIG. 9 is a schematic diagram of a vestibular electrical stimulation apparatus 100A in the second embodiment. In FIG. 9, the description of the components that coincide with those of the vestibular electrical stimulation apparatus 100 in FIG. 1 is not repeated.

In the second embodiment, the vestibular electrical stimulation apparatus 100A is not connected to an external apparatus such as the game apparatus 200 and is used independently. The vestibular electrical stimulation apparatus 100A includes a switch 140 instead of the input interface 120. The switch 140 may be, for example, a mechanical button or a touch panel.

In the second embodiment, when riding in a conveyance such as a ship, an airplane, a train, or a vehicle where the user Ur1 may develop motion sickness, the user Ur1 attaches the electrodes E1 and E2 and operates the switch 140. Based on the operation on the switch 140, the control circuit 110 applies the current WN1 to the electrodes E1 and E2.

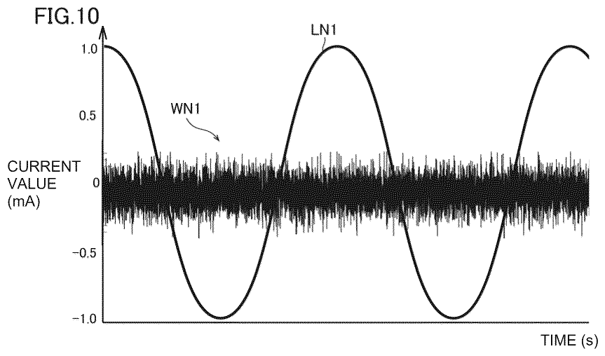

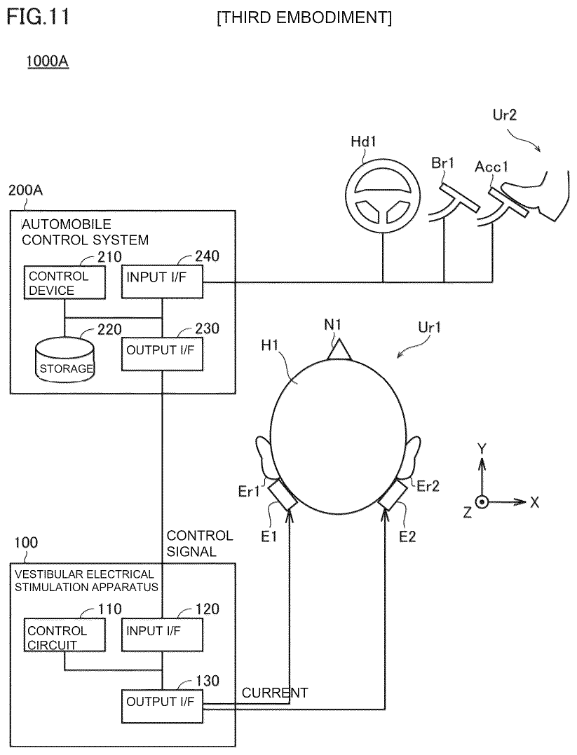

FIG. 10 is a diagram for describing the current WN1 that is less than the perception threshold and a current LN1 that is greater than or equal to the perception threshold in the second embodiment. In the second embodiment, based on the operation on the switch 140, the control circuit 110 applies the current LN1 for making the user Ur1 feel a pseudo-acceleration, in addition to the current WN1. The current LN1 corresponds to a “second current” in the present disclosure.

The current LN1 is an alternating current having a so-called “cradle effect”. The cradle effect is an effect for promoting sleep by providing the user Ur1 with a moderate rocking sensation. The current LN1 is applied to the user Ur1, so that the user Ur1 is provided with reciprocating pseudo-accelerations as the moderate rocking sensation. This promotes sleep of the user Ur1 in the second embodiment. Since motion sickness is improved by sleep, the suppression of the development of motion sickness and early recovery can be promoted more effectively in the second embodiment.

The current LN1 is a current perceivable by the user Ur1. The current LN1 has a larger current density than the current WN1. The current LN1 has a frequency of, for example, 0.25 Hz. As illustrated in FIG. 10, the control circuit 110 applies the current LN1 and the current WN1 in the same period. That is, the control circuit 110 simultaneously applies the current LN1 and the current WN1.

Thus, also in the second embodiment, the current LN1 having a greater current density than the current WN1 can promote sleep of the user Ur1 while the application of the current WN1 reduces the attention of the brain to the vestibular sensation and alleviates the symptoms of motion sickness. In the example of the second embodiment, the configuration in which the current LN1 having the cradle effect is applied together with the current WN1 has been described. However, the control circuit 110 may apply the current WN1 alone, or may alternately apply the current WN1 and the current LN1 instead of simultaneously applying the current WN1 and the current LN1. In the example of the second embodiment, the example in which the user Ur1 is riding in a ship has been described. However, the conveyance in which the user Ur1 rides may be other conveyances such as an airplane, a train, and a vehicle.

### THIRD EMBODIMENT

In the second embodiment, the example in which the current WN1 is applied in the period for which the user Ur1 is riding in a conveyance has been described. In a third embodiment, a configuration in which a current perceivable by the user Ur1 is applied in accordance with stopping, a right turn, a left turn, or the like of a conveyance will be described.

FIG. 11 is a schematic diagram of a control system 1000A in the third embodiment. In FIG. 11, the description of the components that coincide with those of the control system 1000 in FIG. 1 is not repeated.

The vestibular electrical stimulation apparatus 100 in FIG. 11 is connected to an automobile control system 200A instead of the game apparatus 200. The automobile control system 200A is, for example, an ECU (Electronic Control Unit). The automobile control system 200A acquires operation input values for a steering wheel Hd1, a brake Br1, and an accelerator Acc1 via the input interface 240. In the third embodiment, a user Ur2 illustrated in FIG. 11 is a driver, and the user Ur1 is a passenger other than the driver.

The automobile control system 200A detects a turning direction of the vehicle in which the user Ur1 rides, in accordance with the operation input values for the steering wheel Hd1, the brake Br1, and the accelerator Acc1. The automobile control system 200A provides the user Ur1 with a pseudo-acceleration in the same direction as the turning direction of the vehicle. That is, when the vehicle in which the user Ur1 rides actually turns right, a current is applied such that the electrode E2 serves as the positive terminal as illustrated in FIG. 3.

In the third embodiment, to provide the user Ur1 with a pseudo-acceleration in the same direction as the turning direction of the vehicle, the automobile control system 200A detects the orientation of the head H1 of the user Ur1. The orientation of the head H1 may be detected, for example, based on information from a sensor attached to the head H1 or a camera in the vehicle.

Providing a pseudo-acceleration in the same direction as the turning direction of the vehicle enhances a vestibular sensation of turning right in the vestibular sensation input to the brain of the user Ur1. This allows the vision information indicating that the vehicle is actually turning to match the vestibular sensation. This leads to the solution of the error between the vision and the vestibular sensation. In the third embodiment, the current for providing a pseudo-acceleration in the same direction as the turning direction of the vehicle corresponds to a “third current” for making a user feel the pseudo-acceleration. In the third embodiment, the period in which the current for providing a pseudo-acceleration in the same direction as the turning direction of the vehicle is applied corresponds to a “second period” in the present disclosure.

In the third embodiment, the current WN1 is applied in a period other than the period in which the pseudo-acceleration in the same direction as the turning direction of the vehicle is provided. For example, in the third embodiment, the current WN1 is applied in a period for which the vehicle is stationary. This can reduce the attention of the brain of the user Ur1 to the vestibular sensation, and alleviate the symptoms of motion sickness also in the third embodiment.

### Appendix 1

A vestibular electrical stimulation apparatus includes:
an electrode configured to provide electrical stimulation to a vestibular organ of a user;
an output circuit configured to apply a current to the electrode;
a control circuit configured to control the current to be applied by the output circuit; and
an input circuit configured to receive a control signal from an external apparatus, wherein
the control circuit is configured to apply a first current, based on the control signal received by the input circuit, the first current being a noise current.

### Appendix 2

The vestibular electrical stimulation apparatus according to Appendix 1, wherein the control circuit is configured to apply the first current when the control signal does not include electrical stimulation data that defines characteristics of the current to be applied to the electrode.

### Appendix 3

The vestibular electrical stimulation apparatus according to Appendix 1 or 2, wherein the first current has a current density that is less than a predetermined threshold and is less than a perception threshold value of the user.

### Appendix 4

The vestibular electrical stimulation apparatus according to any one of Appendices 1 to 3, wherein the first current is white noise.

### Appendix 5

The vestibular electrical stimulation apparatus according to any one of Appendices 1 to 3, wherein the first current is an alternating current having a first frequency.

### Appendix 6

The vestibular electrical stimulation apparatus according to any one of Appendix 1 to 5, wherein
the control circuit is configured to
   cause a second current to be applied, the second current being an alternating current for making the user feel a pseudo-acceleration, and
   the second current has a current density that is greater than a current density of the first current.

### Appendix 7

The vestibular electrical stimulation apparatus according to Appendix 6, wherein the control circuit is configured to apply the first current and the second current in a same period.

### Appendix 8

The vestibular electrical stimulation apparatus according to any one of Appendices 1 to 5, further including:
an input circuit configured to receive a control signal from an external apparatus, wherein
the control circuit is configured to
   acquire, based on the control signal received by the input circuit, a first period in which the first current is applied and a second period in which a third current is applied, the third current being a current for making the user feel a pseudo-acceleration.

### Appendix 9

A control system including:
the vestibular electrical stimulation apparatus according to Appendix 8; and
the external apparatus to be connected to the vestibular electrical stimulation apparatus.

### Appendix 10

The control system according to Appendix 9, wherein
the external apparatus includes:
   an operation circuit configured to receive an operation of the user;
   a display device configured to provide vision information to the user; and
   a control device configured to cause the display device to provide the vision information, the vision information representing an object that is placed in a virtual space and moves in accordance with an operation input value received by the operation circuit, and
the control device is configured to output the control signal to the vestibular electrical stimulation apparatus, based on a movement of the object in the virtual space, the control signal causing the third current to be applied to the electrode.

### Appendix 11

The control system according to Appendix 10, wherein the first period is a non-operation period for which the object is not operable.

### Appendix 12

The control system according to Appendix 9, wherein
the external apparatus includes:
   an operation circuit configured to receive an operation on a steering wheel, a brake, and an accelerator of a vehicle; and
   a control device configured to acquire an operation input value received by the operation circuit, and
the control device is configured to output the control signal to the vestibular electrical stimulation apparatus, in accordance with a movement of the vehicle, the control signal causing the third current to be applied to the electrode.

### Appendix 13

A control method for use in a vestibular electrical stimulation apparatus,
the vestibular electrical stimulation apparatus including:
   an electrode configured to provide electrical stimulation to a vestibular organ of a user;
   an output circuit configured to apply a current to the electrode; and
   an input circuit configured to receive a control signal from an external apparatus,
the control method including:
a step of determining whether a current timing is in a first period in which a first current is applied, the first current being a noise current; and
a step of applying the first current when it is determined that the current timing is in the first period.

The embodiments disclosed herein should be construed as being illustrative and non-restrictive in every respect. The scope of the present invention is defined by the claims, rather than the description above, and is intended to include every modification within the meaning and scope equivalent to the claims.

### REFERENCE SIGNS LIST

100, 100A vestibular electrical stimulation apparatus
110 control circuit
120, 240 input interface
130, 230 output interface
140 switch
200 game apparatus
200A automobile control system
210 control device
220 storage
1000, 1000A control system
A1, A2 operation period
Ab1, Ab2 button
Ac1 acceleration
Acc1 accelerator
B1, B2 non-operation period
Br1 brake
Cr1 vehicle
Cs1 cross control
Ct1 controller
D1 right direction
Df1, Df2, Df3 sensory input
E1, E2 electrode
Ef1, Ef2 support
Er1 left ear
Er2 right ear
H1 head
Hd1 steering wheel
LN1, WN1 current
N1 nose
S1 display device
Ur1, Ur2 user
VG1 goggles

1. A vestibular electrical stimulation apparatus comprising:
an electrode configured to provide electrical stimulation to a vestibular organ of a user;
an output circuit configured to apply a current to the electrode;
a control circuit configured to control the current to be applied by the output circuit; and
an input circuit configured to receive a control signal from an external apparatus, wherein
the control circuit is configured to apply a first current, based on the control signal received by the input circuit, the first current being a noise current.

2. The vestibular electrical stimulation apparatus according to claim 1, wherein the control circuit is configured to apply the first current when the control signal indicates an absence electrical stimulation data that defines characteristics of the current to be applied to the electrode.

3. The vestibular electrical stimulation apparatus according to claim 1, wherein the first current has a current density that is less than a predetermined threshold and is less than a perception threshold value of the user.

4. The vestibular electrical stimulation apparatus according to claim 1, wherein the first current is white noise.

5. The vestibular electrical stimulation apparatus according to claim 1, wherein the first current is an alternating current having a first frequency.

6. The vestibular electrical stimulation apparatus according to claim 1, wherein
the control circuit is configured to
cause a second current to be applied, the second current being an alternating current for making the user feel a pseudo-acceleration, and
the second current has a current density that is greater than a current density of the first current.

7. The vestibular electrical stimulation apparatus according to claim 6, wherein the control circuit is configured to apply the first current and the second current in a same period.

8. The vestibular electrical stimulation apparatus of claim 6, wherein the control circuit is configured to cause the output circuit to apply the first current and the second current in different, non-overlapping periods.

9. The vestibular electrical stimulation apparatus of claim 6, wherein the second current has a frequency of approximately 0.25 Hz.

10. The vestibular electrical stimulation apparatus according to claim 1, wherein
the control circuit is configured to, based on the control signal received by the input circuit,
identify a first period for applying the first current; and
identify a second period for applying a third current, the third current being a current for making the user feel a pseudo-acceleration.

11. The vestibular electrical stimulation apparatus of claim 10, wherein the first period is a non-operation period in which a user is not operating a virtual object or vehicle.

12. The vestibular electrical stimulation apparatus of claim 10, wherein the third current is applied to generate a vestibular sensation that corresponds to a visual stimulus presented to the user by the external apparatus.

13. A control system comprising:
the vestibular electrical stimulation apparatus according to claim 10; and
the external apparatus to be connected to the vestibular electrical stimulation apparatus.

14. The control system according to claim 13, wherein
the external apparatus includes:
an operation circuit configured to receive an operation of the user;
a display device configured to provide vision information to the user; and
a control device configured to cause the display device to provide the vision information, the vision information representing an object in a virtual space and that moves in accordance with an operation input value received by the operation circuit, and
the control device is configured to output the control signal to the vestibular electrical stimulation apparatus, based on a movement of the object in the virtual space, the control signal causing the third current to be applied to the electrode.

15. The control system according to claim 14, wherein the first period is a non-operation period for which the object is not operable by the user.

16. The control system according to claim 13, wherein
the external apparatus includes:
an operation circuit configured to receive an operation of a vehicle; and
a control device configured to acquire an operation input value received by the operation circuit, and
the control device is configured to output the control signal to the vestibular electrical stimulation apparatus, in accordance with a movement of the vehicle, the control signal causing the third current to be applied to the electrode.

17. The control system of claim 16, wherein the first period is a period when the vehicle is stationary.

18. A control method for use in a vestibular electrical stimulation apparatus that includes an electrode for stimulating a vestibular organ of a user, an output circuit, and an input circuit, the control method comprising:
receiving, at the input circuit, a control signal from an external apparatus;
determining, based on the control signal, whether a current timing corresponds to a first period;
in response to determining the current timing corresponds to the first period, controlling the output circuit to apply a first current to the electrode, the first current being a noise current and
otherwise control the output circuit to apply a current to the electrode for providing electrical stimulation to the vestibular organ that is perceivable by the user.

19. The control method of claim 18, wherein the first current is continuously applied.

20. A control system for alleviating motion sickness, the system comprising:
an external apparatus including:
an operation circuit configured to receive an operation of a user;
a display device configured to provide vision information to the user; and
a control device configured to generate a control signal based on an operation input value received by the operation circuit; and
a vestibular electrical stimulation apparatus including:
an electrode configured to be placed in proximity to a vestibular organ of the user;
an input circuit configured to receive the control signal from the control device of the external apparatus; and
a control circuit configured to cause a first current to be applied to the electrode during a first period identified from the control signal, the first current being a noise current.
